(19) United States
(12) Patent Application Publication
(10) Pub. No.: US 2013/0231276 A1
(43) Pub. Date: Sep. 5, 2013

(54) Combination Of An Aminoacyl-tRNA Synthetase Inhibitor With A Further Antibacterial Agent For Attenuating Multiple Drug Resistance

(71) Applicant: BROWN UNIVERSITY, Providence, RI (US)

(72) Inventors: Jason K. Sello, Providence, RI (US); James J. Vecchione, Smithfield, RI (US)

(73) Assignee: Brown University, Providence, RI (US)

(21) Appl. No.: 13/862,136

(22) Filed: Apr. 12, 2013

Related U.S. Application Data
(63) Continuation of application No. PCT/US2011/057119, filed on Oct. 20, 2011.
(60) Provisional application No. 61/405,771, filed on Oct. 22, 2010.
(60) Provisional application No. 61/426,289, filed on Dec. 22, 2010.

(51) Int. Cl. A61K 31/422; A61K 31/165; A61K 31/7048; A61K 38/14

(52) U.S. Cl. CPC A61K 31/422; A61K 38/14; A61K 31/165; A61K 31/7048
USPC 514/2.6; 514/2.9; 514/376; 514/628; 514/29; 514/2.7; 514/2.8

(57) ABSTRACT

Bacterial multidrug resistance is attenuated in a subject by administering an aminoacyl-tRNA synthetase inhibitor and an antibacterial agent to the subject, wherein the aminoacyl-tRNA synthetase inhibitor is distinct from the antibacterial agent.

### RELATED APPLICATIONS

This application is a continuation of International Application No. PCT/US2011/057119, which designated the United States and was filed on Oct. 20, 2011, published in English, which claims the benefit of U.S. Provisional Application Nos. 61/405,771, filed Oct. 22, 2010, and 61/426,289, filed on Dec. 22, 2010. The entire teachings of the above applications are incorporated herein by reference.

### BACKGROUND OF THE INVENTION

Many microorganisms have survived for many years by adapting to antimicrobial agents, which permits certain bacteria to resist antibiotics. Bacteria that exhibit multidrug resistance include, for example, Staphylococci, Enterococci, Gonococci, Streptococci, Salmonella and Mycobacterium tuberculosis. Multidrug resistance can lead to illness and death. Thus, there is a need for new and effective methods to attenuate multidrug resistance in a subject.

### SUMMARY OF THE INVENTION

The invention is generally directed to methods of attenuating bacterial multidrug resistance.

In an embodiment, the invention is a method of attenuating bacterial multidrug resistance in a subject, comprising the step of administering an aminoacyl-tRNA synthetase inhibitor and an antibacterial agent to the subject, wherein the aminoacyl-tRNA synthetase inhibitor is distinct from the antibacterial agent and the aminoacyl-tRNA synthetase inhibitor is neither an isoleucyl-tRNA synthetase inhibitor nor a methionyl-tRNA synthetase inhibitor.

In another embodiment, the invention is a method of attenuating resistance to an antibacterial agent that has aminoacyl-tRNA synthetase inhibitor activity in a subject, comprising the step of administering a composition that includes an aminoacyl-tRNA synthetase inhibitor and an antibacterial agent to the subject, wherein the aminoacyl-tRNA synthetase inhibitor is distinct from the antibacterial agent and the aminoacyl-tRNA synthetase inhibitor is neither an isoleucyl-tRNA synthetase inhibitor nor a methionyl-tRNA synthetase inhibitor.

In another embodiment, the invention is a method of treating a subject with a multidrug resistant bacterial infection, comprising the step of administering an aminoacyl-tRNA synthetase inhibitor and an antibacterial agent to the subject, wherein the aminoacyl-tRNA synthetase inhibitor is distinct from the antibacterial agent.

### BRIEF DESCRIPTION OF THE DRAWINGS

FIG. 1 depicts the effect of indolmycin on the chloramphenicol sensitivity of four indolmycin-resistant strains of S. coelicolor.

FIG. 2 depicts the effect of indolmycin on the erythromycin sensitivity of four indolmycin-resistant strains of S. coelicolor.

FIG. 3 depicts the effect of indolmycin on the vancomycin sensitivity of four indolmycin-resistant strains of S. coelicolor.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principal features of this invention can be employed in various embodiments without departing from the scope of the invention.

In an embodiment, the invention is a method of attenuating bacterial multidrug resistance in a subject, comprising the step of administering an aminoacyl-tRNA synthetase inhibitor and an antibacterial agent to the subject, wherein the aminoacyl-tRNA synthetase inhibitor is distinct from the antibacterial agent and the aminoacyl-tRNA synthetase inhibitor is neither an isoleucyl-tRNA synthetase inhibitor nor a methionyl-tRNA synthetase inhibitor.

In another embodiment, the invention is a method of attenuating resistance to an antibacterial agent that has aminoacyl-tRNA synthetase inhibitor activity in a subject, comprising the step of administering a composition that includes an aminoacyl-tRNA synthetase inhibitor and an antibacterial agent to the subject, wherein the aminoacyl-tRNA synthetase inhibitor is distinct from the antibacterial agent and the aminoacyl-tRNA synthetase inhibitor is neither an isoleucyl-tRNA synthetase inhibitor nor a methionyl-tRNA synthetase inhibitor.

“Attenuate,” as used herein with respect to bacterial multidrug resistance, means that the multidrug resistance is reduced, preferably to have a clinically beneficial outcome. Attenuate is also referred to as “decrease” with respect to bacterial multidrug resistance.

“Distinct,” as used herein in reference to the aminoacyl-tRNA synthetase inhibitor and the antibacterial agent used in the methods of the invention, means that the aminoacyl-tRNA synthetase inhibitor and the antibacterial agent are different compounds.

In another embodiment, the invention is a method of treating a subject with a multidrug resistant bacterial infection, comprising the step of administering an aminoacyl-tRNA synthetase inhibitor and an antibacterial agent to the subject, wherein the aminoacyl-tRNA synthetase inhibitor is distinct from the antibacterial agent.

The aminoacyl-tRNA synthetase inhibitor employed in the methods of the invention can be a natural product inhibitor, an analog of a natural product inhibitor, or pharmaceutically acceptable salts. Exemplary natural product inhibitors of aminoacyl-tRNA synthetases include mupirocin, borrelidin, furanomycin, granaticin, indolmycin, ochratoxin A, and cis-pentacin. The aminoacyl-tRNA synthetase inhibitors of the present invention include inhibitors of, for example, at least one member selected from the group consisting of isoleucyl (e.g., mupirocin, furanomycin), leucyl (e.g., granaticin), threonyl (e.g., borrelidin), phenylalanyl (e.g., ochratoxin A), tryptophanyl (e.g., indolmycin, chuangxinmycin), methionyl, prolyl (e.g., cis-pentacin) and lysyl tRNA synthetases.

The aminoacyl-tRNA synthetase inhibitor employed in the methods of the invention can be a tryptophanyl-tRNA synthetase inhibitor (e.g., indolmycin or a pharmaceutically acceptable salt). The antibacterial agent employed in the methods of the invention can be at least one member selected from the group consisting of ampicillin, chloramphenicol, erythromycin, lincomycin, mupirocin and vancomycin or pharmaceutically acceptable salts. In some embodiments, the antibacterial agent employed can be at least one member selected from the streptogramin class of antibiotics.

Aminoacyl-tRNA synthetase inhibitors interfere with the biosynthesis of charged aminoacyl-tRNA substrates for protein synthesis. Many antibiotic resistance determinants are proteins. If a particular charged aminoacyl-tRNA is unavailable, then protein synthesis cannot be completed. Therefore, inhibition of aminoacyl-tRNA synthetases could lead to inhibition of the biosynthesis of resistance determinants. For example, by perturbing the biosynthesis of the tryptophan-rich proteins required for resistance to chloramphenicol, erythromycin and vancomycin, a tryptophanyl-tRNA synthetase inhibitor (e.g., indolmycin, chuangxinmycin) can suppress resistance to chloramphenicol, erythromycin, and vancomycin.

In an embodiment, the aminoacyl-tRNA synthetase inhibitor and the antibacterial agent can be co-administered to the subject. In another embodiment, the aminoacyl-tRNA synthetase inhibitor can be administered prior to administration of the antibacterial agent. In a further embodiment, the antibacterial agent is administered prior to the aminoacyl-tRNA synthetase inhibitor.

The methods of the invention can attenuate bacterial multidrug resistance to at least one bacteria selected from the group consisting of Staphylococci, Enterococci, Gonococci, Streptococci, Salmonella and Mycobacterium tuberculosis. In other embodiments, the methods of the invention can attenuate bacterial multidrug resistance to Streptomyces. In yet another embodiment, the methods of the invention can attenuate resistance to methicillin-resistant Staphylococcus aureus (MRSA).

An “effective amount,” also referred to herein as a “therapeutically effective amount,” when referring to the amount of a compound or composition (e.g., an antibacterial agent, an aminoacyl-tRNA synthetase inhibitor) is defined as that amount, or dose, of a compound or composition that, when administered to a subject, is sufficient for therapeutic efficacy (e.g., an amount sufficient to attenuate multidrug resistance, an amount sufficient to prevent a multidrug resistant bacterial infection).

The methods of the invention can be accomplished by the administration of the compounds of the invention (e.g., compositions including an antibacterial agent, an aminoacyl-tRNA synthetase inhibitor) to a subject (e.g., a human subject) by enteral or parenteral means. The route of administration can be by oral ingestion (e.g., tablet, capsule form) or intramuscular injection of the compound. Other routes of administration can include intravenous, intraarterial, intraperitoneal, or subcutaneous routes, nasal administration, suppositories and transdermal patches. The compounds employed in the methods of the invention can be administered in suitable excipients, including pharmaceutically acceptable salts.

In an embodiment, the compounds (e.g., an antibacterial agent, an aminoacyl-tRNA synthetase inhibitor) employed in the methods of the invention can be administered in a dose of between about 0.01 mg/kg to about 0.1 mg/kg; about 0.001 mg/kg to about 0.01 mg/kg; about 0.001 to about 0.05 mg/kg; about 0.1 mg/kg to about 1 mg/kg body weight; about 1 mg/kg to about 5 mg/kg body weight; or between about 5 mg/kg to about 15 mg/kg body weight.

The compounds can be administered in doses of about 0.1 mg, about 1 mg, about 2 mg, about 2.5 mg, about 5 mg, about 10 mg, about 15 mg, about 20 mg, about 25 mg, about 30 mg, about 40 mg, about 45 mg, about 50 mg, about 60 mg, about 80 mg, 100 mg, about 200 mg, about 250 mg, about 300 mg, about 350 mg, about 400 mg, about 500 mg, about 600 mg, about 700 mg, about 900 mg, about 1000 mg, about 1200 mg, about 1400 mg, about 1600 mg or about 2000 mg, or any combination thereof. The compounds can be administered once a day or multiple (e.g., two, three, four, five) times per day.

### EXEMPLIFICATION

There has been much interest in using combinations of antibacterial drugs to treat multidrug resistant pathogens and/or slow the rate at which drug resistance emerges (1-4). Streptomyces coelicolor is a non-pathogenic relative of the Gram-positive, human pathogen Mycobacterium tuberculosis (5). S. coelicolor is a useful model organism for studies of antibacterial resistance because because it is resistant to several antibiotics, including indolmycin, chloramphenicol, daptomycin, erythromycin, the streptogramins, and vancomycin (6-12). With the exception of daptomycin, the resistance of S. coelicolor to each of these antibiotics has been ascribed to the expression of one or more genes (6, 8-12). We assessed the effect of indolmycin, an antibiotic that inhibits bacterial tryptophanyl-tRNA synthetases (8-10, 13), on the viability of S. coelicolor when used in combination with other antibiotics to which the organism is resistant.

Changes in the susceptibility of S. coelicolor to three clinically used antibacterial drugs (chloramphenicol, erythromycin, and vancomycin) grown in the presence of indolmycin were assessed. The effects of antibiotic combinations were assessed in wild-type S. coelicolor, which has an auxiliary, indolmycin-resistant isoform of tryptophanyl-tRNA synthetase (TrpRS1). We also examined these combinations in three other indolmycin-resistant strains of S. coelicolor—a trpRS2 null strain (B728) (8) and two strains lacking trpRS1 with resistance conferring point mutations in trpRS2 (B734 with TrpRS2 H48N and B735 with TrpRS2 H48Q) (9). These strains were selected because the strong antibacterial activity of indolmycin precluded testing of this hypothesis in the indolmycin-sensitive trpRS1 null strain of S. coelicolor. The effects were measured in terms of changes in the chloramphenicol, erythromycin, and vancomycin MICs of the four strains over a range of sub-lethal indolmycin concentrations.

Indolmycin markedly suppresses the resistance of S. coelicolor to chloramphenicol (a bacteriostatic protein synthesis inhibitor than binds to the ribosome), erythromycin (a macrolide antibiotic), and vancomycin (a glycopeptide antibiotic). The fact that indolmycin suppresses resistance to at least three different antibacterial drugs (each having a different mechanism of action) is notable and potentially valuable. It suggests that indolmycin can be useful even if the bacterium to be killed is resistant to it or other antibiotics, provided that the proteins conferring resistance require tryptophan for their biosynthesis.

Indolmycin can be used to kill multi-drug resistant strains that are sensitive to indolmycin. However, when the inevitable resistance to indolmycin emerges, indolmycin may still be useful as an adjuvant to other antibacterial drugs to which a pathogenic bacterium is resistant. This utility may be due to the ability of indolmycin to suppress other mechanisms of drug resistance by interfering with the biosynthesis of resistance determinants.

Aminoacyl-tRNA synthetase inhibitors have emerged as a useful class of antibacterial drugs. The prototypical member of this class is the clinically used drug mupirocin, which is an inhibitor of isoleucyl-tRNA synthetase. Another aminoacyl-tRNA synthetase inhibitor that has attracted attention is indolmycin. This antibiotic, derived from Streptomyces griseus ATCC 12648, is a competitive inhibitor of bacterial tryptophanyl-tRNA synthetases. Although indolmycin is not presently used in the clinic, its potent activity against methicillin-resistant Staphylococcus aureus (MIC is 0.5 μg/mL) and Helicobacter pylori (MIC is 0.016 μg/mL) has renewed interest in its clinical utility.

Resistance to indolmycin has been reported. Many indolmycin-resistant bacterial species harbor auxiliary isoforms of tryptophanyl-tRNA synthetase that are insensitive to indolmycin. In Streptomyces coelicolor (a non-pathogenic relative of Mycobacterium tuberculosis), transcription of the gene encoding an auxiliary, indolmycin-resistant isoform of tryptophanyl-tRNA synthetase (trpRS1) is induced by indolmycin. The induced expression of trpRS1 was confirmed by analytical two-dimensional gel electrophoresis and peptide-mass fingerprinting of soluble protein isolated from S. coelicolor grown in media containing 40 μg/mL indolmycin. In the same experiment, the abundance of several proteins was notably reduced. These observations were intriguing because S. coelicolor is highly resistant to indolmycin (MIC is about>500 μg/mL) and did not exhibit any apparent growth defects in the experiment. Proteins whose synthesis was negatively affected by indolmycin have multiple tryptophan residues.

Indolmycin may reduce the availability of charged tryptophanyl-tRNA for protein synthesis even though the resistance gene trpRS1 is expressed. TrpRS1 is weakly inhibited by the antibiotic (Ki=about 900 nM) (19). Indolmycin may perturb the synthesis of antibacterial drug resistance determinants with multiple tryptophan residues and consequently affect the susceptibility of S. coelicolor to the corresponding antibacterial drugs. S. coelicolor is a useful model organism for studies of multi-drug resistant bacteria because it is resistant to several clinically used antibacterial drugs, including ampicillin, chloramphenicol, erythromycin, lincomycin, mupirocin, and vancomycin, via multiple genetic determinants. Erythromycin, chloramphenicol, and vancomycin resistance determinants have multiple tryptophan residues.

Streptomyces coelicolor strains were grown on DIFCO Nutrient Agar supplemented with indolmycin (0-100 μg/mL) and one of the following antibacterial agents: chloramphenicol (10-80 μg /mL), erythromycin (1-110 μg/mL), and vancomycin (30-140 μg/mL). The strains were grown on the media at 30° C. for 48 hours, after which point growth was assessed visually.

Vancomycin was purchased from Sigma Chemical Co. and a 50 mg/ml stock solution was created by dissolving the solid vancomycin in dH₂0. Chloramphenicol was purchased from Sigma Chemical Co., and a 25 mg/ml stock solution was created by dissolving the chloramphenicol in 100% ethanol. Erythromycin was purchased from Sigma Chemical Co., and a 50 mg/ml stock solution was created by dissolving the erythromycin in 100% ethanol. Ochratoxin A and borrelidin can be purchased from Sigma Chemical Co. Indolmycin was chemically synthesized according to established procedures as described, for example, by Hasuoka, A., et. al., Chem. Pharm. Bull. 49:1604-1608 (2001).

A 50 mg/mL (active enantiomer) stock solution of indolmycin was prepared by dissolving the indolmycin in DMSO. The concentrations of each stock solution reflect the actual concentration of the pharmacologically active substance. Aliquots of the stock solutions were added directly to molten, sterile DIFCO Nutrient Agar prior to solidification. Streptomyces coelicolor spores were directly spread onto the surface of each DNA plate and the plates were incubated at 30° C. for about 48 hours. Growth was assessed visually after the incubation period. Minimal inhibitory concentrations (MICs) reflect the drug concentrations at which no Streptomyces coelicolor growth was observed.

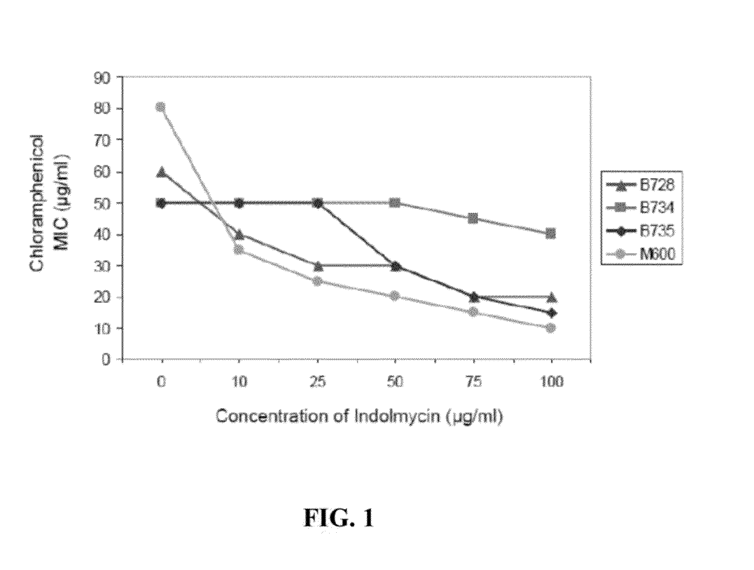

Initially, the impact of indolmycin on chloramphenicol resistance in S. coelicolor was tested. Chloramphenicol is an antibiotic used in the treatment of eye infections. In S. coelicolor, chloramphenicol resistance is conferred by two major facilitator superfamily efflux pumps, Cm1R1 and Cm1R2 (7). Since each of these membrane proteins has seven tryptophan residues, indolmycin may perturb their biosynthesis and, thus affect chloramphenicol susceptibility in S. coelicolor. The chloramphenicol MIC of all four strains was reduced in media supplemented with indolmycin (Table 1, FIG. 1). The most dramatic effect was observed in wild-type S. coelicolor, where the chloramphenicol MIC was reduced 10-fold.

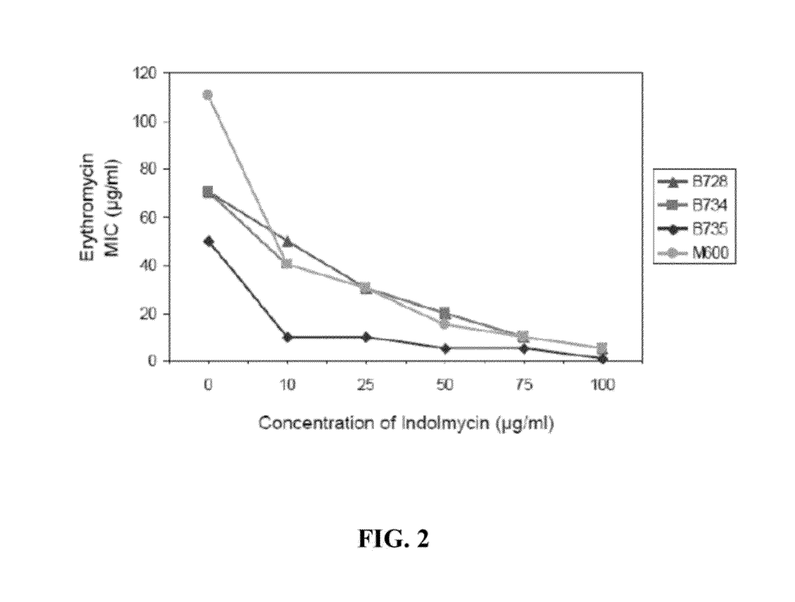

Next, the impact of indolmycin on erythromycin resistance in S. coelicolor was tested. Erythromycin is a macrolide antibiotic and its semi-synthetic derivatives are used in clinical medicine for treatment of a variety of bacterial infections. The erythromycin resistance phenotype of S. coelicolor has been ascribed to a gene (SC06090) encoding a glycosyl transferase with 9 tryptophan residues (5). Indolmycin may negatively affect the biosynthesis of the erythromycin resistance determinant. Co-administration of indolmycin with erythromycin profoundly affected the viability of all four strains (Table 2, FIG. 2). Strain B735 was found to be 50-times more sensitive to erythromycin in the presence of about 100 μg/mL indolmycin.

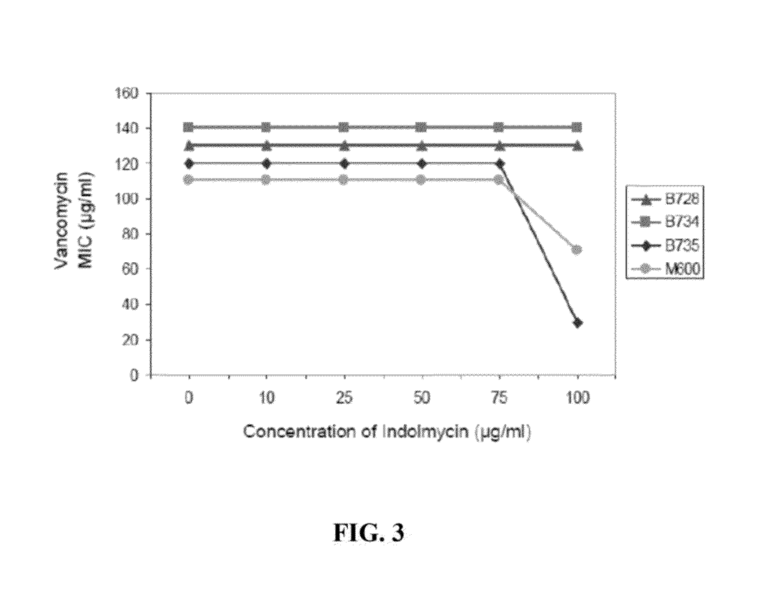

Finally, the effect of indolmycin on vancomycin resistance in S. coelicolor was assessed. Vancomycin is a glycopeptide antibiotic that is widely known as the “drug of last resort” in the treatment of infections caused by multidrug-resistant bacteria. As such, vancomycin resistance is a concern and has been the subject of extensive study (14). S. coelicolor has the characteristic vancomycin resistance cassette consisting of genes encoding a two-component regulatory system and enzymes that convert D-Ala-D-Ala termini of muropeptides to D-Ala-D-Lac (12). All 7 of the gene products encoded by this cassette have tryptophan residues (e.g., the membrane-bound histidine kinase VanS has 4 tryptophan residues and the D-Ala-D-Ala dipeptidase VanX has 6 tryptophan residues). Given the tryptophan content of the van gene products, indolmycin may make the S. coelicolor strains susceptible to vancomycin. Interestingly, indolmycin affected vancomycin susceptibility in only two of the four strains (Table 3, FIG. 3). In media supplemented with about 100 μg/mL indolmycin, strains M600 and B735 were 1.6- and 4-times more sensitive to vancomycin, respectively.

In conclusion, indolmycin can suppress three different drug resistance phenotypes of a multi-drug resistant bacterium. Although it has potential as an antibacterial drug (15-18), indolmycin is not clinically used at present. In strains where resistance to indolmycin is innate or in strains where it emerged by point mutations (8-10), indolmycin could be used as an adjuvant to other antibacterial drugs to which a bacterium is resistant. An explanation for the apparent indolmycin-induced changes in the drug MICs may be partial inhibition of indolmycin-resistant tryptophanyl-tRNA synthetases, which could perturb the biosynthesis of resistance determinants that are rich in tryptophan residues. This explanation is supported by the observation that the indolmycin Kᵢ of the resistant tryptophanyl-tRNA synthetase (TrpRS1) in S. coelicolor is 900 nM (19); thus, this isoform is likely to be inhibited by the antibiotic. Further, proteins in S. coelicolor mediating chloramphenicol, erythromycin, and vancomycin resistance are rich in tryptophan residues (Table 4).

While differing degrees of resistance to indolmycin may explain the extent to which the antibiotic affects the antibacterial MICs of specific strains, differences in gene expression, enzymatic activity, and/or protein stability could be used to explain the finding that indolmycin affects the MICs of certain drugs more than others. In any case, the suppression of multiple drug resistance phenotypes by indolmycin is noteworthy in the context of combination therapies (1-4). Combinations of antibacterial drugs to treat multidrug resistant pathogens and/or slow the rate at which drug resistance emerges have been reported (2, 3, 10, 15). For example, for AUGMENTIN®, a β-lactam inhibitor (potassium clavulanate) is used in conjunction with a β-lactam drug (20). As an adjuvant, indolmycin has a broader spectrum than potassium clavulanate because it presumably perturbs the biosynthesis of several resistance determinants. Since many drug resistance determinants (e.g., membrane-bound efflux pumps) in bacteria are rich in tryptophan residues, tryptophanyl-tRNA synthetase inhibitors like indolmycin could be used at sub-lethal concentrations to suppress multi-drug resistance phenotypes.

As described herein, indolmycin in combination with at least one antibacterial agent, can attenuate bacterial multidrug resistance to indolmycin, chloramphenicol, erythromycin and vancomycin. Methods of the invention may have the advantage for use if the bacteria to which drug resistance exists or will develop requires particular amino acids for resistance. For example, a tryptophanyl-tRNA synthetase inhibitor can be used in combination with an antibacterial agent to attenuate bacterial multidrug resistance against bacterial that require tryptophan for biosynthesis of proteins for multidrug resistance or growth.

TABLE 1
The Effect of Indolmycin on Chloramphenicol Sensitivity

| | Chloramphenicol MIC (μg/ml) | | | | | |
|---|---|---|---|---|---|---|
| | 0 μg/ml | 10 μg/ml | 25 μg/ml | 50 μg/ml | 75 μg/ml | 100 μg/ml |
| Strain | Indolmycin | Indolmycin | Indolmycin | Indolmycin | Indolmycin | Indolmycin |
| M600 | 80 | 35 | 25 | 20 | 15 | 10 |
| B728 | 60 | 40 | 30 | 30 | 20 | 20 |
| B734 | 50 | 50 | 50 | 50 | 45 | 40 |
| B735 | 50 | 50 | 50 | 30 | 20 | 15 |

All Streptomyces strains were grown on Difco Nutrient Agar at 30° C. The MICs were assessed after 48 hours.

TABLE 2
The Effect of Indolmycin on Erythromycin Sensitivity

| | Erythromycin MIC (μg/ml) | | | | | |
|---|---|---|---|---|---|---|
| | 0 μg/ml | 10 μg/ml | 25 μg/ml | 50 μg/ml | 75 μg/ml | 100 μg/ml |
| Strain | Indolmycin | Indolmycin | Indolmycin | Indolmycin | Indolmycin | Indolmycin |
| M600 | 110 | 40 | 30 | 15 | 10 | 5 |
| B728 | 70 | 50 | 30 | 20 | 10 | 5 |
| B734 | 70 | 40 | 30 | 20 | 10 | 5 |
| B735 | 50 | 10 | 10 | 5 | 5 | 1 |

All Streptomyces strains were grown on Difco Nutrient Agar at 30° C. The MICs were assessed after 48 hours.

TABLE 3
The Effect of Indolmycin on Vancomycin Sensitivity

| | Vancomycin MIC (μg/ml) | | | | | |
|---|---|---|---|---|---|---|
| | 0 μg/ml | 10 μg/ml | 25 μg/ml | 50 μg/ml | 75 μg/ml | 100 μg/ml |
| Strain | Indolmycin | Indolmycin | Indolmycin | Indolmycin | Indolmycin | Indolmycin |
| M600 | 110 | 110 | 110 | 110 | 110 | 70 |
| B728 | 130 | 130 | 130 | 130 | 130 | 130 |
| B734 | 140 | 140 | 140 | 140 | 140 | 140 |
| B735 | 120 | 120 | 120 | 120 | 120 | 30 |

All Streptomyces strains were grown on Difco Nutrient Agar at 30° C. The MICs were assessed after 48 hours.

TABLE 4
Erythromycin, chloramphenicol, and vancomycin resistance
determinants in S. coelicolor and their tryptophan content

| Gene | | Number of |
|---|---|---|
| Number | Description | Tryptophan Residues |
| SCO7526 | cmlR1, chloramphenicol efflux | 7 |
| | pump | |
| SCO7662 | cmlR2, chloramphenicol efflux | 7 |
| | pump | |
| SCO6090 | Putative erythromycin | 9 |
| | glycosyltransferase | |
| SCO3589 | vanS, probable two component | 4 |
| | sensor kinase | |
| SCO3596 | vanX, probable D-alanine; | 6 |
| | D-alanine dipeptidase | |

### REFERENCES

1. Chait, R., et. al., Nature 446:668-671 (2007).

2. Golan, D. E. et al. (eds). Principles of Pharmacology: The Pathophysiologic Basis of Drug Therapy. (Lippincott Williams & Wilkins, Philadelphia, 2005)

3. Keith, C. T., et. al., Nat. Rev. Drug Discov. 4:71-8 (2005).

4. Torella, J. P., et. al., PLoS Computational Biology 6: e1000796 (2010).

5. Bentley, S. D., et. al., Streptomyces coelicolor. Nature 417:141-147 (2002).

6. Kieser, T., et. al., Practical Streptomyces Genetics. Norwich: John Innes Foundation (2000).

7. Vecchione, J. J., et. al., Antimicrob. Agents Chemother. 53:4673-4677 (2009).

8. Vecchione, J. J., et. al., J. Bacteriol. 190:6253-6257 (2008).

9. Vecchione, J. J. et. al., Antimicrob. Agents Chemother. 53:3972-3980 (2009).

10. Vecchione, J. J. et. al., J. Bacteriol. 192:3565-3573 (2010).

11. Folcher, M., et. al., J. Biol. Chem. 276:1479-1485 (2001).

12. Hong, H.-J., et. al., Mol. Microbiol. 59: 1107-1121 (2004).

13. Werner, R. G., et. al., Eur. J. Biochem. 68: 1-3 (1976).

14. Hong, H. J., et. al., Adv. Exp. Med. Biol. 631: 200-13 (2008).

15. Kim, S., et. al., Appl. Microbiol. Biotechnol. 61:278-288 (2003).

16. Hurdle, J. G., et. al., J. Antimicrob. Chemother. 54:549-552 (2004).

17. Hurdle, J. G., et. al., Antimicrob. Agents Chemother. 49:4821-4833 (2005).

18. Kanamaru, T., et. al., Antimicrob. Agents Chemother. 45:2455-2459 (2001).

19. Kitabatake, M., K. et. al., J. Biol. Chem. 277:23882-23887 (2002).

20. Stein, G. E. et. al., Clin. Pharmacy 3:591-599 (1984).

The teachings of all of the references cited herein are hereby incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of attenuating bacterial multidrug resistance in a subject, comprising the step of administering at least one aminoacyl-tRNA synthetase inhibitor and at least one antibacterial agent to the subject, wherein the aminoacyl-tRNA synthetase inhibitor is distinct from the antibacterial agent and the aminoacyl-tRNA synthetase inhibitor is neither an isoleucyl-tRNA synthetase inhibitor nor a methionyl-tRNA synthetase inhibitor.

2. The method of claim 1, wherein the aminoacyl-tRNA synthetase inhibitor is a tryptophanyl-tRNA synthetase inhibitor.

3. The method of claim 2, wherein the tryptophanyl-tRNA synthetase inhibitor is indolmycin.

4. The method of claim 1, wherein the antibacterial agent is at least one member selected from the group consisting of ampicillin, chloramphenicol, erythromycin, lincomycin, mupirocin and vancomycin.

5. The method of claim 1, wherein the aminoacyl-tRNA synthetase inhibitor and the antibacterial agent are co-administered to the subject.

6. The method of claim 1, wherein the aminoacyl-tRNA synthetase inhibitor is administered prior to administration of the antibacterial agent.

7. The method of claim 1, wherein the antibacterial agent is administered prior to the aminoacyl-tRNA synthetase inhibitor.

8. The method of claim 1, wherein administration of the aminoacyl-tRNA synthetase inhibitor and the antibacterial agent attenuate bacterial multidrug resistance to at least one bacteria selected from the group consisting of Staphylococci, Enterococci, Gonococci, Streptococci, Salmonella and Mycobacterium tuberculosis.

9. The method of claim 8, wherein the Staphylococci is methicillin-resistant Staphylococcus aureus.

10. A method of treating a subject with a multidrug resistant bacterial infection, comprising the step of administering an aminoacyl-tRNA synthetase inhibitor and an antibacterial agent to the subject, wherein the aminoacyl-tRNA synthetase inhibitor is distinct from the antibacterial agent.

11. The method of claim 10, wherein the aminoacyl-tRNA synthetase inhibitor is at least one member selected from the group consisting of tryptophanyl-tRNA synthetase inhibitor, threonyl-tRNA synthetase inhibitor and phenylalanyl-tRNA synthetase inhibitor.

12. The method of claim 11, wherein the aminoacyl-tRNA synthetase inhibitor is a tryptophanyl-tRNA synthetase inhibitor.

13. The method of claim 12, wherein the aminoacyl-tRNA synthetase inhibitor includes at least one member selected from the group consisting of indolmycin and chuangxinmycin.

14. The method of claim 10, wherein the antibacterial agent is at least one member selected from the group consisting of streptogramin, ampicillin, chloramphenicol, erythromycin, lincomycin, mupirocin and vancomycin.

15. A method of attenuating resistance to an antibacterial agent that has aminoacyl-tRNA synthetase activity in a subject, comprising the step of administering an aminoacyl-tRNA synthetase inhibitor and an antibacterial agent to the subject, wherein the aminoacyl-tRNA synthetase inhibitor is distinct from the antibacterial agent and the aminoacyl-tRNA synthetase inhibitor is neither an isoleucyl-tRNA synthetase inhibitor nor a methionyl-tRNA synthetase inhibitor.

16. The method of claim 15, wherein the aminoacyl-tRNA synthetase inhibitor is at least one member selected from the group consisting of tryptophanyl-tRNA synthetase inhibitor, threonyl-tRNA synthetase inhibitor and phenylalanyl-tRNA synthetase inhibitor.

17. The method of claim 16, wherein the aminoacyl-tRNA synthetase inhibitor is a tryptophanyl-tRNA synthetase inhibitor.

18. The method of claim 15, wherein the antibacterial agent is at least one member selected from the group consisting of indolmycin, mupirocin, chuangxinmycin, ochratoxin A and borrelidin.

19. The method of claim 15, wherein the administration of the aminoacyl-tRNA synthetase inhibitor and the additional antibacterial agent attenuate bacterial multidrug resistance to at least one bacteria selected from the group consisting of Staphylococci, Enterococci, Gonococci, Streptococci, Salmonella and Mycobacterium tuberculosis.

20. The method of claim 19, wherein the Staphylococci is methicillin-resistant Staphylococcus aureus.
